(19) United States
(12) Patent Application Publication
(10) Pub. No.: US 2022/0264922 A1
(43) Pub. Date: Aug. 25, 2022

(54) NATURAL VANILLIN COMPOSITIONS

(71) Applicant: RHODIA OPERATIONS, Aubervilliers (FR)

(72) Inventor: Laurent GAREL, Lyon (FR)

(21) Appl. No.: 17/627,152

(22) PCT Filed: Jul. 30, 2020
(86) PCT No.: PCT/EP2020/071485

§ 371 Date: Jan. 14, 2022

(30) Foreign Application Priority Data
Jul. 30, 2019 (FR) FR1908643

(51) Int. Cl. A23L 27/20; A61K 8/34; A61K 47/10; C11D 3/50; C11D 3/20; A23L 27/10; A23K 10/30; A23K 20/111

(52) U.S. Cl. CPC A23L 27/204; A61K 8/347; A61K 47/10; C11D 3/50; C11D 3/2072; A23L 27/11; A23K 10/30; A23K 20/111; A61K 2236/37; A23V 2002/00

(57) ABSTRACT

The present invention relates to natural vanillin compositions comprising at least one compound chosen from 4-((4-hydroxy-3-methoxybenzyl)oxy)-3-methoxybenzaldehyde and 4-hydroxy-3-(4-hydroxy-3-methoxybenzyl)-5-methoxybenzaldehyde. The invention also relates to a process for obtaining natural vanillin compositions according to the invention.

The present application claims the priority of the patent application filed on Jul. 30, 2019 in France under the number 1908643 and the content of which is incorporated in full by way of reference.

### FIELD OF THE INVENTION

The present invention relates to natural vanillin compositions comprising at least one compound chosen from 4-((4-hydroxy-3-methoxybenzyl)oxy)-3-methoxybenzaldehyde and 4-hydroxy-3-(4-hydroxy-3-methoxybenzyl)-5-methoxybenzaldehyde. The invention also relates to a process for obtaining natural vanillin compositions according to the invention.

### PRIOR ART

Vanillin, or 4-hydroxy-3-methoxybenzaldehyde, may be obtained by various methods known to a person skilled in the art, in particular by the following three routes:
A “natural” route based on a biotechnological process comprising in particular the culturing of a microorganism capable of enabling the bioconversion of a fermentation substrate into vanillin. Such a process in which the fermentation substrate is ferulic acid is in particular known from application EP 0 885 968. The ferulic acid used may be of several origins, in particular derived from rice bran or corn bran.
A “synthetic” route comprising conventional chemical reactions starting from guaiacol that does not involve a microorganism. This process results in the preparation of a vanillin termed synthetic vanillin.
Lastly, vanillin may also be prepared according to a route described as “bio-based” in which the vanillin is obtained from eugenol or lignin; mention may in particular be made of the documents U.S. Pat. No. 2,745,796, DE 1132113 and the paper entitled “Preparation of lignin from wood dust as vanillin source and comparison of different extraction methods” by Azadbakht et al. in the International Journal of Biology and Biotechnology, 2004, Vol. 1, No. 4, pp 535-537.

When the “natural route” is used for the industrial production of vanillin, several side reactions may occur leading to the formation of numerous by-products which may affect the organoleptic properties of the natural vanillin. The chemical synthesis of one of the by-products thereof is described in particular in EP 2763947. As a result, the preparation of a natural vanillin composition having the correct properties is complex, in particular in terms of color or organoleptic properties. The present invention is aimed at the efficient and controlled preparation of natural vanillin compositions that have a known and controlled content of by-products.

### BRIEF DESCRIPTION

Thus, a first subject of the present invention relates to a composition comprising natural vanillin and further comprising at least one compound chosen from 4-((4-hydroxy-3-methoxybenzyl)oxy)-3-methoxybenzaldehyde and 4-hydroxy-3-(4-hydroxy-3-methoxybenzyl)-5-methoxybenzaldehyde.

Another aspect relates to a process for preparing a natural vanillin composition according to the invention, comprising a step (a) of fermenting ferulic acid and optionally a step (b) of purifying the natural vanillin composition obtained at the end of step (a).

Lastly, the present invention relates to the use of a natural vanillin composition as a flavoring in the field of human food and animal feed, in pharmaceuticals, or as a fragrance in the cosmetics, perfumery and detergent industries.

### DETAILED DESCRIPTION

In the context of the present invention, and unless otherwise indicated, the expression “between . . . and . . . ” includes the limits. Unless otherwise indicated, the percentages and ppm are percentages and ppm by weight.

In the context of the present invention, and unless otherwise indicated, the term “ppm” means “parts per million”. This unit represents a fraction by weight: 1 ppm=1 mg/kg.

In the context of the present invention, and unless indicated otherwise, the expression “natural vanillin” may denote a flavoring substance according to Article 9.2(c) of Regulation (EC) No 1334/2008, that is to say a flavoring substance obtained by physical, enzymatic or microbiological processes from materials of vegetable, animal or microbiological origin, either as in the raw state of the material or after processing for human consumption by one or more of the traditional food preparation processes. A natural flavoring substance corresponds to a substance which is naturally present and has been identified in nature. The definitions given by the regulations in force in other countries or regions may also be applied. Preferably, the term “natural vanillin” according to the present invention denotes vanillin obtained by a biotechnological process, comprising the culturing of a microorganism capable of enabling the conversion of a fermentation substrate into vanillin. According to the invention, the microorganism may be wild or be genetically modified, in particular in order to improve the yield of vanillin or to prevent the formation of by-products. Very preferentially, it may be a process for the fermentation of ferulic acid, as described in patent application EP 0 885 968. The ferulic acid may be of any origin, in particular derived from rice bran, beet pulp, wheat bran, bagasse, sugarcane or corn, in particular corn bran or corn fibers, in particular from starch factories or nixtamalization units.

According to a first aspect, the present invention relates to a composition comprising natural vanillin and further comprising at least one compound chosen from 4-((4-hydroxy-3-methoxybenzyl)oxy)-3-methoxybenzaldehyde and 4-hydroxy-3-(4-hydroxy-3-methoxybenzyl)-5-methoxybentaldehyde.

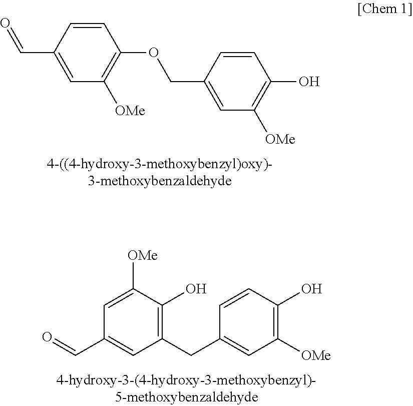 [SMILES: COc1cc(COc2ccc(C=O)cc2OC)ccc1O ; COc1cc(Cc2cc(C=O)cc(OC)c2O)ccc1O]

According to a particular aspect, the composition according to the present invention comprises 4-((4-hydroxy-3-methoxybenzyl)oxy)-3-methoxybenzaldehyde and 4-hydroxy-3-(4-hydroxy-3-methoxybenzyl)-5-methoxybenzaldehyde.

According to a preferred aspect, the composition according to the present invention further comprises at least one compound chosen from vanillyl alcohol, vanillic acid, acetovanillone, guaiacol and vinylguaiacol. For example, the composition according to the present invention may comprise acetovanillone and/or vinylguaiacol.

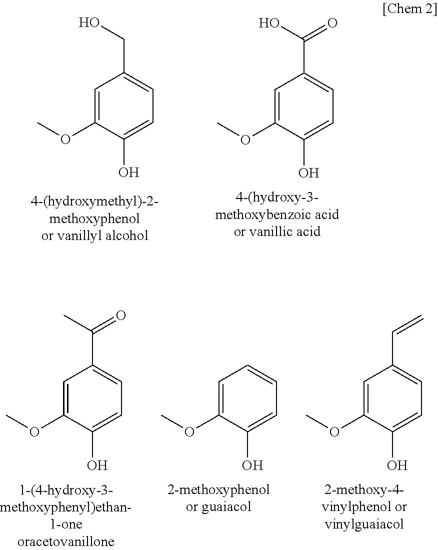

Preferably, the composition according to the present invention may further comprise at least one compound chosen from vanillyl alcohol, vanillic acid and acetovanillone.

Advantageously, the composition according to the present invention exhibits a natural vanillin titer of greater than or equal to 90%, preferentially greater than 95%, very preferentially greater than or equal to 99%. According to a particular aspect, the natural vanillin titer is greater than or equal to 99.5%.

According to a preferred aspect, the content by weight of 4-((4-hydroxy-3-methoxybenzyl)oxy)-3-methoxybenzaldehyde, relative to the total weight of the composition, is less than or equal to 10 000 ppm, preferably less than or equal to 8000 ppm, preferentially less than or equal to 7000 ppm, and very preferentially less than or equal to 5000 ppm.

According to another preferred aspect, the content by weight of 4-((4-hydroxy-3-methoxybenzyl)oxy)-3-methoxybenzaldehyde, relative to the total weight of the composition, is greater than or equal to 10 ppm, preferably greater than or equal to 200 ppm, preferentially greater than or equal to 500 ppm, and very preferentially greater than or equal to 100 ppm.

Advantageously, the content by weight of 4-((4-hydroxy-3-methoxybenzyl)oxy)-3-methoxybenzaldehyde, relative to the total weight of the composition, is between 600 ppm and 2500 ppm.

According to a preferred aspect, the content by weight of 4-hydroxy-3-(4-hydroxy-3-methoxybenzyl)-5-methoxybenzaldehyde, relative to the total weight of the composition, is less than or equal to 5000 ppm, preferably less than or equal to 2500 ppm, preferentially less than or equal to 1000 ppm, and very preferentially less than or equal to 900 ppm.

According to another preferred aspect, the content by weight of 4-hydroxy-3-(4-hydroxy-3-methoxybenzyl)-5-methoxybenzaldehyde, relative to the total weight of the composition, is greater than or equal to 10 ppm, preferably greater than or equal to 100 ppm, preferentially greater than or equal to 200 ppm, and very preferentially greater than or equal to 300 ppm.

Advantageously, the content by weight of 4-hydroxy-3-(4-hydroxy-3-methoxybenzyl)-5-methoxybenzaldehyde, relative to the total weight of the composition, is between 400 ppm and 800 ppm.

According to the present invention, the content by weight of vanillyl alcohol, relative to the total weight of the composition, is less than or equal to 4000 ppm, preferably less than or equal to 2000 ppm, very preferentially less than or equal to 1000 ppm, preferably less than or equal to 800 ppm, preferentially less than or equal to 600 ppm, and very preferentially less than or equal to 500 ppm. According to the present invention, the content by weight of vanillyl alcohol, relative to the total weight of the composition, is greater than or equal to 1 ppm, preferably greater than or equal to 5 ppm, preferentially greater than or equal to 10 ppm, and very preferentially greater than or equal to 20 ppm.

Advantageously, the content by weight of vanillyl alcohol, relative to the total weight of the composition, is between 50 ppm and 250 ppm.

According to the present invention, the content by weight of vanillic acid, relative to the total weight of the composition, is less than or equal to 1000 ppm, preferably less than or equal to 700 ppm, preferentially less than or equal to 500 ppm, and very preferentially less than or equal to 200 ppm. According to the present invention, the content by weight of vanillic acid, relative to the total weight of the composition, is greater than or equal to 1 ppm, preferably greater than or equal to 5 ppm, preferentially greater than or equal to 10 ppm, and very preferentially greater than or equal to 20 ppm.

Advantageously, the content by weight of vanillic acid, relative to the total weight of the composition, is between 50 ppm and 100 ppm.

According to the present invention, the content by weight of acetovanillone, relative to the total weight of the composition, is less than or equal to 1000 ppm, preferably less than or equal to 800 ppm, preferentially less than or equal to 600 ppm, and very preferentially less than or equal to 500 ppm. According to the present invention, the content by weight of acetovanillone, relative to the total weight of the composition, is greater than or equal to 0.1 ppm, preferably greater than or equal to 5 ppm, preferentially greater than or equal to 10 ppm, and very preferentially greater than or equal to 20 ppm.

Advantageously, the content by weight of acetovanillone, relative to the total weight of the composition, is between 50 ppm and 400 ppm.

According to the present invention, the content by weight of guaiacol, relative to the total weight of the composition, is less than or equal to 100 ppm, preferably less than or equal to 50 ppm, preferentially less than or equal to 40 ppm, and very preferentially less than or equal to 20 ppm. According to the present invention, the content by weight of guaiacol, relative to the total weight of the composition, is greater than or equal to 0.01 ppm, preferably greater than or equal to 0.1 ppm, preferentially greater than or equal to 1 ppm, and very preferentially greater than or equal to 10 ppm. According to another alternative of the present invention, the vanillin composition does not comprise guaiacol.

According to the present invention, the content by weight of vinylguaiacol, relative to the total weight of the composition, is less than or equal to 100 ppm, preferably less than or equal to 50 ppm, preferentially less than or equal to 40 ppm, and very preferentially less than or equal to 20 ppm.

According to the present invention, the content by weight of vinylguaiacol, relative to the total weight of the composition, is greater than or equal to 0.01 ppm, preferably greater than or equal to 0.1 ppm, preferentially greater than or equal to 1 ppm, and very preferentially greater than or equal to 10 ppm. According to another alternative of the present invention, the vanillin composition does not comprise vinylguaiacol, in particular the vinylguaiacol content is below the detection limits of liquid chromatography methods.

Advantageously, the composition according to the present invention is weakly colored. Preferably, the natural vanillin composition has a color of less than or equal to 400 Hazen, preferably of less than or equal to 200 Hazen, more preferentially of less than or equal to 100 Hazen, very preferentially of less than or equal to 50 Hazen and even more preferentially of less than or equal to 20 Hazen. The color is measured in a 10% by weight ethanolic solution. The color measurements are performed with a Konica Minolta CM-5 spectrophotometer using a cuvette with an optical path length of 10 mm.

The natural vanillin composition according to the present invention may be in crystalline or amorphous, preferably crystalline, form. The natural vanillin composition may be formed as a powder, crystals, beads, prills or flakes.

Advantageously, the natural vanillin composition according to the present invention has an organoleptic profile which is compliant, in particular in terms of visual appearance, texture, taste or fragrance. Advantageously, the composition according to the present invention does not exhibit false notes. In particular, the organoleptic profile of the composition according to the present invention is at least equivalent to the organoleptic profile of natural vanillin extracted from pods. The natural vanillin composition according to the present invention is able to contain other compounds. These compounds present in said composition are associated with the process for preparing natural vanillin. Mention may be made in particular, without however being exhaustive, of 4-(3-hydroxy-4-methoxybenzyl)-2-methoxyphenol, 4,4′-methylenebis(2-methoxyphenol), ferulic acid, and vanillin dimers and/or trimers.

Another aspect relates to a process for preparing a natural vanillin composition according to the invention, characterized in that it comprises a step (a) of fermenting ferulic acid and optionally a step (b) of purifying the crude natural vanillin composition obtained at the end of step (a).

Step (a): According to the invention, step (a) of the process is a step of fermenting ferulic acid for the production of a crude natural vanillin composition.

A crude natural vanillin composition refers to a composition obtained at the end of the ferulic acid fermentation step. In particular, the crude vanillin composition may be obtained after treatment of the aqueous fermentation medium. For example, the fermentation medium may be separated from the biomass, and then the aqueous phase may be extracted with an appropriate solvent. Thus, the crude natural vanillin composition may be obtained at the end of the ferulic acid fermentation step indirectly. Specifically, during the production of natural vanillin, a liquid composition comprising natural vanillin, impurities and a large amount of solvent, typically a food-grade solvent, such as, for example, ethyl acetate, is generally recovered.

The fermentation may be carried out as indicated in document EP 0 885 968. Fermentation is generally carried out in an aqueous medium.

Preferably, at the end of step (a) the weight concentration of natural vanillin is between 0.5% and 60%, preferentially between 5% and 40% and even more preferentially between 10% and 35% relative to the weight of the crude natural vanillin composition.

The crude natural vanillin composition used in step (a) may comprise at least one compound chosen from 4-((4-hydroxy-3-methoxybenzyl)oxy)-3-methoxybenzaldehyde and 4-hydroxy-3-(4-hydroxy-3-methoxybenzyl)-5-methoxybenzaldehyde. The crude vanillin composition may further comprise at least one compound chosen from vanillyl alcohol, vanillic acid, acetovanillone, guaiacol and vinylguaiacol.

Step (b): Step (b) is an optional step of purifying the composition obtained at the end of step (a). In one variant, step (b) is not optional.

In general, step (b) may comprise a liquid-liquid extraction as described in document EP 2 791 098; preferably, the liquid-liquid extraction is carried out using a solvent compatible with food-processing applications.

Step (b) may also comprise at least one crystallization step. According to another alternative, the purification step may comprise an extraction with supercritical CO₂.

Lastly, according to another alternative, step (b) may comprise a step in which the crude natural vanillin is evaporated as described in document EP 2 948 424 or a stripping step as described in document W02018/146210.

According to a particular aspect of the present invention, step (b) is a step of evaporation in a wiped film evaporator or in a falling film evaporator. Advantageously, this step allows the removal of compounds less volatile than vanillin that are present in the crude natural vanillin composition obtained at the end of step (a). The less-volatile compounds are preferentially chosen from vanillyl alcohol, vanillic acid, acetovanillone, and heavy compounds.

The term “heavy compounds” refers to compounds that are less volatile than vanillin under the temperature and pressure conditions considered. Among the heavy impurities, mention may be made of dimers and trimers of vanillin (that is to say compounds exhibiting a backbone having two or three phenyl groups, respectively), and ferulic acid.

This embodiment is particularly advantageous in that it makes it possible to purify a crude natural vanillin composition obtained at the end of step (a) and allows the preparation of a natural vanillin composition according to the present invention. The content of impurities is particularly well controlled and the natural vanillin composition is very weakly colored.

The process according to the present invention is capable of limiting the content of 4-((4-hydroxy-3-methoxybenzyl)oxy)-3-methoxybenzaldehyde and 4-hydroxy-3-(4-hydroxy-3-methoxybenzyl)-5-methoxybenzaldehyde.

Furthermore, this embodiment makes it possible to avoid a step of stripping in the presence of an entraining gas and/or a vaporized liquid. Thus, this process is interesting from an industrial viewpoint in that it is economically viable.

The temperature of the heat-transfer fluid at which step (b) is carried out is generally greater than or equal to 130° C., preferentially greater than or equal to 145° C. In general, the temperature of the heat-transfer fluid is less than or equal to 230° C., preferentially less than or equal to 180° C.

The pressure at which step (b) is carried out is generally greater than or equal to 0.4 mbar, preferentially greater than or equal to 1 mbar. In general, the pressure is less than or equal to 75 mbar, preferentially less than or equal to 8 mbar and more preferentially less than or equal to 4 mbar.

Advantageously, the weight concentration of natural vanillin in the crude natural vanillin composition used in step (b) is greater than or equal to 10%, preferentially greater than or equal to 20%, more preferentially greater than or equal to 30%, and even more preferentially greater than or equal to 50%, and very preferentially greater than or equal to 70%, relative to the total weight of the crude natural vanillin composition. The process may comprise a step of concentrating the crude natural vanillin composition.

According to a preferred aspect, step (b) is carried out in the presence of at least one technical adjuvant, for example a fluidifying agent. Thus, according to a specific embodiment of the present invention, a fluidifying agent authorized by regulations on foodstuffs may be added to the natural vanillin. The fluidifying agent may be added before step (b), in particular between step (a) and step (b). According to another embodiment, the fluidifying agent may be added during step (b).

The fluidifying agent may be chosen from white mineral oils, in particular food-grade white mineral oils, polyethylene glycols, in particular having an average molecular weight of between 200 and 3000 g/mol, preferably of between 600 and 1500 g/mol, or castor oil.

Preferentially, step (b) is carried out under an inert atmosphere, in particular under argon, nitrogen or mixtures thereof. Step (b) may also be carried out under oxygen-depleted air. In general, the oxygen content is less than or equal to 1%.

The evaporator makes it possible to recover a gas effluent containing the evaporated natural vanillin composition, which is then condensed so as to recover a condensate of the natural vanillin composition according to the invention.

Advantageously, the natural vanillin titer is greater than or equal to 90%, very preferentially greater than 95%, and even more preferentially greater than or equal to 99%. According to a particular aspect, the vanillin titer is greater than or equal to 99.5%.

Advantageously, the natural vanillin condensate obtained at the end of the natural vanillin purification step (b) is weakly colored. Advantageously, the natural vanillin condensate obtained at the end of step (b) is a natural vanillin composition according to the present invention.

Advantageously, this natural vanillin condensate, obtained at the end of step (b), exhibits organoleptic properties which are compliant, and at least equivalent to those of natural vanillin extracted from pods or of synthetic vanillin.

According to a particular aspect, the purification step (b) does not comprise a stripping step.

According to a particular aspect, the process of the invention does not comprise a step consisting in melting the natural vanillin composition prior to the step of evaporation in a falling film or wiped film evaporator. According to a particular aspect, the natural vanillin composition is not crystallized between step (a) and step (b).

The process may also comprise an optional step (c) of forming the natural vanillin composition obtained at the end of step (b), preferably by crystallization, more preferentially by crystallization in a solvent identified by the FEMA GRAS™ program. In a preferred embodiment, the natural vanillin composition is crystallized or recrystallized from water or from a water/alcohol, in particular ethanol, mixture.

The process according to the invention may be carried out according to a continuous operation or according to a batchwise operation.

The invention also relates to a natural vanillin composition capable of being obtained according to the process described according to the present invention.

Lastly, the present invention relates to the use of a natural vanillin composition as a flavoring in the field of human food and animal feed, in pharmaceuticals, or as a fragrance in the cosmetics, perfumery and detergent industries.

The examples below are intended to illustrate the invention without, however, limiting it.

Should the disclosure of patents, patent applications and publications cited herein by way of reference contradict the description of the present application to the extent that it risks rendering a term uncertain, the present description shall prevail.

### EXAMPLE

Example 1: A crude natural vanillin composition is prepared from a fermentation carried out according to the procedure described in application EP 0 885 968. Besides the solvent, the crude vanillin composition comprised the compounds in table 1.

TABLE 1

| | |
|---|---|
| Natural vanillin | 21% by weight |
| Vanillyl alcohol | 2.7% by weight |
| Vanillic acid | 0.2% by weight |
| Acetovanillone | Not measured |
| 4-((4-hydroxy-3-methoxybenzyl)oxy)-3- | Not detected |
| methoxybenzaldehyde | |
| 4-hydroxy-3-(4-hydroxy-3-methoxybenzyl)- | Not detected |
| 5-methoxybenzaldehyde | |

Example 2: A crude natural vanillin composition obtained according to the process of application EP 0 885 968 is subjected to purification by vacuum evaporation in the presence of castor oil, in a wiped film evaporator.

Properties of the natural vanillin composition according to the invention:

TABLE 1

| | | |
|---|---|---|
| Natural vanillin | 99.7% | by weight |
| 4-((4-hydroxy-3-methoxybenzyl)oxy)-3- | 1000 | ppm |
| methoxybenzaldehyde | | |
| 4-hydroxy-3-(4-hydroxy-3-methoxybenzyl)- | 400 | ppm |
| 5-methoxybenzaldehyde | | |

| | |
|---|---|
| Color (Hazen) (10% by weight ethanolic | 20 |
| solution) | |

1-10. (canceled)

11. A composition comprising natural vanillin and further comprising at least one compound chosen from 4-((4-hydroxy-3-methoxybenzyl)oxy)-3-methoxybenzaldehyde and 4-hydroxy-3-(4-hydroxy-3-methoxybenzyl)-5-methoxybenzaldehyde, the composition exhibiting a natural vanillin titer of greater than or equal to 90%.

12. The composition of claim 11, further comprising at least one compound chosen from vanillyl alcohol, vanillic acid, acetovanillone, guaiacol and vinylguaiacol, preferably chosen from vanillyl alcohol, vanillic acid and acetovanillone.

13. The composition of claim 11, wherein the natural vanillin titer is greater than 95%.

14. The composition of claim 11, wherein the content by weight of 4-((4-hydroxy-3-methoxybenzyl)oxy)-3-methoxybenzaldehyde, relative to the total weight of the composition, is less than or equal to 10 000 ppm and greater than or equal to 10 ppm.

15. The composition of claim 11, wherein the content by weight of 4-hydroxy-3-(4-hydroxy-3-methoxybenzyl)-5-methoxybenzaldehyde, relative to the total weight of the composition, is less than or equal to 5000 ppm and greater than or equal to 10 ppm.

16. The composition of claim 11, comprising a color of less than or equal to 400 Hazen.

17. A process for preparing the composition of claim 11, the process comprising a step (a) of fermenting ferulic acid and optionally a step (b) of purifying the natural vanillin composition obtained at the end of step (a).

18. The process claim 17, wherein step (b) is a step of evaporation in a wiped film evaporator or of evaporation in a falling film evaporator, optionally carried out in the presence of a technical adjuvant.

19. The process as claimed in claim 18, wherein step (b) comprises a liquid/liquid extraction or an extraction with supercritical CO₂.

20. A method for flavoring, the method comprising using the composition of claim 11 as a flavoring in the field of human food and animal feed, in pharmaceuticals, or as a fragrance in the cosmetics, perfumery and detergent industries.
